(19) United States
(12) Patent Application Publication
(10) Pub. No.: US 2024/0225903 A9
(43) Pub. Date: Jul. 11, 2024

(54) HEARING PROTECTION DEVICES

(71) Applicant: MINUENDO AS, Oslo (NO)

(72) Inventors: Olav KVALØY, Oslo (NO); Tom A. TRONES, Oslo (NO); Odd PETERSEN, Oslo (NO)

(21) Appl. No.: 17/769,494

(22) PCT Filed: Oct. 16, 2020
(86) PCT No.: PCT/GB2020/052623

§ 371 Date: Apr. 15, 2022

(30) Foreign Application Priority Data
Oct. 18, 2019 (GB) 1915152.1

(51) Int. Cl. A61F 11/14; A61F 11/12; H04R 25/00

(52) U.S. Cl. CPC A61F 11/145; A61F 11/12; H04R 25/30; H04R 2460/15

(57) ABSTRACT

A hearing protection device (500) arranged to provide an acoustic barrier is disclosed. The hearing protection device (500) includes a microphone (502) arranged to measure sound behind the acoustic barrier. The device (500) determines whether a sound level characteristic of the sound measured by the microphone (502) is above a predetermined threshold. When the sound level characteristic measured by the microphone (502) is above the predetermined threshold, the device (500) compares the sound level characteristic of the sound behind and outside the acoustic barrier to determine an attenuation of the device (500). If the determined attenuation is below a cut-off level of attenuation, the device (500) provides an alert.

This invention relates to hearing protection, in particular determining the actual effectiveness of a hearing protection device.

In a variety of industries, for example construction and performance arts, employees are subjected to high amplitude noises. When exposure to high amplitude noises occurs over an extended period of time, it can result in permanent damage to the function of the ear i.e. to an individual's hearing. As such, appropriate protection against these high amplitude noises is important to reduce the health risks of working in such environments.

Whilst protection, in the form of earplugs (both active and passive) and over-the-ear defenders, is available to reduce the health risk associated with exposure to high amplitude noises, this protection may be incorrectly fitted or not worn as appropriate. This may be because individuals find such protection uncomfortable or inconvenient to wear or have a flawed understanding of the level of risk they face in their current environment. Incorrectly fitted protection may result in inadequate attenuation or no attenuation being provided by the protection equipment. Whilst it is known that this is a problem, given that it is dependent on the compliance of many different individuals, it can be difficult to determine the extent of the problem.

The fit of the hearing protection may also be liable to change over time. For example, an earplug may become at least partially dislodged from a correctly fitted position as an individual using the earplug performs physical tasks. Therefore, a hearing protection device which is initially correctly fitted may become incorrectly fitted over a period of time and may then provide unreliable protection against hearing loss.

In previously known earplug devices, mechanisms for determining the attenuation of the device have been proposed to help initially customise the device so that it provides a threshold level of protection. This may include moulding the shape of the device to the ear canal of an individual. However, these mechanisms do not offer any assistance with the problem of devices not being properly fitted or not being used when they should.

When viewed from a first aspect, the invention provides a system for evaluating an attenuation of a hearing protection device, the system comprising:
a hearing protection device arranged to provide an acoustic barrier;
a microphone arranged to measure sound behind the acoustic barrier;
a sound generator arranged to generate a test sound outside the acoustic barrier with a known sound characteristic; and
a processor arranged to compare repeatedly during normal use the test sound generated by the sound generator with the measured sound behind the acoustic barrier to determine an attenuation of the hearing protection device; the system being arranged to provide an alert if the determined attenuation is below a cut-off level of attenuation.

Thus it will be seen that in accordance with the invention, the effectiveness of an hearing protection device can be determined regularly when the device is being used by an individual. An alert can then be provided, e.g. to an individual using the device or a central controller, if the determined attenuation of the hearing protection device is below the cut-off level of attenuation. This may help individuals in noisy environments to be more certain of the attenuation, and therefore protection, provided by the hearing protection device. Providing the alert (when the determined attenuation of the hearing protection device is below the cut-off level of attenuation) may incentivise an individual to improve the fit of their hearing protection device in order to improve the attenuation of sound by the hearing protection device. This may result in a reduced risk of damage to an individual's hearing. More generally it provides the possibility for general monitoring of the level of compliance and the ability to allow immediate intervention when protection is not being properly used.

Measuring the sound behind the acoustic barrier could mean measuring any sound characteristic, but preferably means measuring a sound level characteristic which is a measure of how loud the sound is, e.g. a sound pressure. Optionally, the sound is measured at a given frequency or over a given range of frequencies.

By having a sound generator to generate the test sound, the assessment of whether the protection device is properly fitted can be made at any time without relying on external factors such as a particular level of sound being present. This allows greater control. Carrying out the assessment based on a known sound characteristic may also enable greater accuracy to be achieved.

The cut-off level will typically be what is determined to provide adequate hearing protection. It will typically be slightly lower than the theoretical maximum level of attenuation that the hearing device can provide so as to account for manufacturing tolerances, a normal level of variation in fitting e.g. as a result of the user's having differently shaped ears, measurement tolerances etc.

In a set of embodiments, the hearing protection device is designed for insertion into an ear canal. An example of a device for insertion into an ear canal is an earplug. In such devices the acoustic barrier is typically formed in the outer part of the ear canal. The device may be correctly fitted when the device is fully inserted into the ear canal. However if the device in not fully inserted, the level of attenuation it provides will typically be reduced.

In another set of embodiments the hearing protection device comprises a pair of over-the-ear defenders in which the acoustic barrier is formed by providing a seal against the user's head around their respective outer ears. The system may comprise multiple hearing protection devices. For example, for very noisy environments the system may comprise a device for insertion into an ear canal and a pair of over-the-ear defenders. In such a system the attenuation from outside the outermost acoustic barrier to behind the innermost acoustic barrier would be the relevant one to measure.

In embodiments in which the hearing protection device comprises a pair of over-the-ear defenders, the device may be correctly fitted when the over-the-ear defenders are positioned to bear against the user's head and enclose their outer ears the respective acoustic barriers. Again however, if the device is not properly fitted, e.g. where the headband is incorrectly adjusted or a part of the outer ear is trapped beneath the cushioned seal such that the cushioned seal does not seal properly against the user's head, the level of attenuation provided will be reduced.

In a further set of embodiments the hearing protection device comprises a pair of devices arranged for insertion into respective ear canals and connected by a common connecting structure. The common connecting structure may be suitable for resting around an individual's shoulders and/or neck—e.g. in the form of a collar. Providing a pair of devices for insertion in the ear canals on a common connecting structure may encourage users to keep the hearing protection device on their person even, for example, when hearing protection is not required but without the discomfort which may be experienced with bulky ear defenders. The risk of losing or forgetting to implement the hearing protection devices may thereby be reduced.

Conveniently, the hearing protection device comprises the microphone. Whilst the microphone should be located acoustically behind the acoustic barrier provided by the hearing protection device in order to measure sound behind the acoustic barrier, it will be appreciated that in practice it may be convenient to provide the microphone within the structure which forms the acoustic barrier. In such arrangements a channel can be provided from the microphone to behind the acoustic barrier.

Embodiments may be envisaged in which the sound generator is provided separately of the hearing protection device, e.g. in a designated test environment or by another device carried by the user. For example, a smart phone may comprise an application configured as a sound generator to generate a test sound with known sound characteristics. In a set of embodiments however, the sound generator is provided on the hearing protection device. As with the microphone it may be practical to provide it within the structure which forms the acoustic barrier and to provide a suitable channel so that the sound is provided outside the acoustic barrier. Alternatively the sound generator may be provided on the aforementioned common connecting structure.

In a set of embodiments, the processor is provided on the hearing protection device. This may be beneficial in providing a device which is self-contained without requiring a user to have access to or remember to carry other equipment. In exemplary embodiments the processor is provided on the aforementioned common connecting structure. However, the Applicant has also appreciated that there may be embodiments in which the processor is provided separately to the hearing protection device. In a set of embodiments, the system comprises a mobile device wherein the mobile device provides the processor. For example, the mobile device may comprise an application configured to compare during normal use the test sound produced by the sound generator (which may also be provided by the mobile device as set out above) with the measured sound behind the acoustic barrier to determine an attenuation of the hearing protection device. In such embodiments the hearing protection device will typically need to be arranged to pass (e.g. wirelessly) information regarding the sound measured by the microphone.

The provision of a test sound with a known sound characteristic and the comparison of the test sound produced by the sound generator with the measured sound behind the acoustic barrier may be considered to be an attenuation test. The attenuation test may be carried out repeatedly over the lifetime of the device by carrying it out at least once during every period in which the protection device should be used—e.g. every shift for an industrial worker. Advantageously however the attenuation test is carried out repeatedly during a given period of use. Repeating the attenuation test during the period over which the individual is using the device can better help to detect, and alert the individual to, the attenuation provided by the hearing protection device being below the cut-off level of attenuation at times after it is initially fitted. This may for example help to discourage removal of the protection device after a period of time (which the user may have become accustomed to doing), to remind the user to replace it properly if it has been removed legitimately (e.g. in a break or when working in a low noise environment) or to refit and/or adjust the protection device if the device has become displaced over a period of time.

Repetition of the attenuation test may be achieved in any suitable and desired way. In a set of embodiments, the attenuation test is carried out periodically e.g. every 30 minutes, every hour etc. By determining the attenuation provided by the hearing device at regular time intervals, compliance can be ensured in a straightforward way and safe behaviour may be reinforced.

In a set of embodiments, the system carries out the attenuation test in response to an event. The event may be any suitable and desired event. The event may be any suitable and desired signal. For example, the event may be receipt of a test trigger signal. For example, such a signal could be transmitted by a central unit to the system. This signal may be received by the processor or a receiver in the hearing protection device. The central unit may be a remote unit for management of multiple individuals using the system. This may, for example, allow a manager to initiate an attenuation test to check a hearing protection device is providing protection against damage to the user's hearing or the central unit may have a pre-defined schedule or otherwise determine that as test is required. Equally however the test trigger signal could be a short-range signal transmitted in, or at the entrance to, a potentially hazardous zone.

In another, potentially overlapping, set of embodiments the event may be provided by an individual using the system. For example, the system may comprise a user interface such as a button for initiating an attenuation test. This helps the user implementing the hearing protection device to control when the attenuation test is performed. For example, a user may notice that noise levels have increased, that they are entering a potentially hazardous zone or it may simply be that they have been instructed to carry out the test at a certain point—e.g. at the beginning of a shift or returning from a break.

In embodiments where the system comprises a separate mobile device, this may be arranged to initiate the attenuation test, either automatically or in response to interaction from the user.

In another set of embodiments (again potentially overlapping), the system may comprise a position determining means, arrangement or subsystem. This would allow the system itself to determine its location and the event could comprise the user entering a particular location. Such position determination could, for example use the Global Positioning System and/or comprise an indoor positioning system.

It will of course be appreciated that an attenuation test might be initiated in response to multiple triggers according to one or more predefined algorithms. For example a test might be normally be carried out only when entering specific zones, but with background tests being carried out if none has been carried out for at least an hour.

Carrying out attenuation tests only when needed may help to extend battery life and so reduce how often it is necessary to re-charge or allow smaller and so lighter batteries to be used, which may be important to the comfort of the user. In a set of embodiments, the microphone is arranged to measure a sound level characteristic behind the acoustic barrier and the system is arranged to carry out an attenuation test when said sound level characteristic behind the acoustic barrier exceeds a threshold internal sound level characteristic. In other words the sound level characteristic behind the acoustic barrier exceeding the threshold internal sound level characteristic constitutes the previously-described event. An example of a sound level characteristic which may be measured is a sound pressure, optionally at a given frequency or over a given frequency range. If the sound level characteristic behind the acoustic barrier exceeds such a threshold, this may suggest that either: the hearing protection device is incorrectly fitted and therefore not attenuating the sound from outside the acoustic barrier sufficiently as it is supposed to; and/or the sound levels outside the acoustic barrier are at such a high level that the hearing protection device is not providing sufficient attenuation of the sound to reduce the risk of damage to hearing despite being correctly fitted. The latter might be deduced after the attenuation test is carried out without necessitating the aforementioned alert to be provided. In this case a different alert may be provided to indicate that the user needs to move or employ additional protection. Advantageously, no interaction is required by the individual or by another user (e.g. a manager) for the attenuation test to be carried out. The attenuation test therefore be automatic.

In a set of embodiments, the system further comprises a second microphone arranged to measure sound outside the acoustic barrier. In a set of such embodiments the hearing protection device comprises the second microphone. Whilst the second microphone should be located acoustically outside the acoustic barrier provided by the hearing protection device in order to measure sound outside the acoustic barrier, it will be appreciated that in practice it may be convenient to provide the second microphone within the structure which forms the acoustic barrier. In such arrangements a channel can be provided between the second microphone and the outside of the acoustic barrier. The second microphone could be located on a common printed circuit board (PCB) with the first microphone.

In another set of such embodiments the second microphone is provided separately of the hearing protection device, e.g. by another device carried by the user such as a mobile device or smart phone. As previously discussed, this may help to minimise the cost of the system. In embodiments in which the system comprises a common connecting structure, the second microphone may be provided on the common connecting structure.

In a set of embodiments the second microphone is arranged to measure the test sound generated by the sound generator. This may help to increase the certainty in the sound characteristics of the test sound, as the sound characteristics of the test sound generated can be measured by the second microphone. This may result in a more accurate determination of the attenuation provided by the hearing protection device. In a set of embodiments therefore, the processor is arranged to compare the measured sound behind the acoustic barrier to the measured sound outside of the acoustic barrier when the sound generator is operative.

Additionally or alternatively, the second microphone may be used to determine the general sound levels outside of the acoustic barrier in the local environment not necessarily when the sound generator is operative i.e. the sound levels an individual would be subjected to without implementing the hearing protection device. A sound level characteristic outside of the acoustic barrier reaching a threshold may constitute the previously mentioned event which is arranged to initiate the attenuation test. Therefore, in a set of embodiments, the system is arranged to carry out an attenuation test when a measured sound level characteristic outside the acoustic barrier exceeds a threshold. As before, the sound level characteristic is preferably sound pressure, optionally at a given frequency or over a give range of frequencies. In such embodiments, the attenuation test may be initiated when the sound levels in the local environment become sufficiently loud that damage may be caused to an individual's hearing if the hearing protection device is not fitted correctly. Carrying out attenuation tests only when needed may help to extend battery life and so reduce how often it is necessary to re-charge or allow smaller and so lighter batteries to be used, which may be important to the comfort of the user. For example, the threshold may be between 75-100 dB, e.g. between 80-95 dB, e.g. 90 dB.

In a set of embodiments, the system is arranged to carry out the attenuation test when a difference between measured sound outside the acoustic barrier and measured sound behind the acoustic barrier exceeds a threshold.

In situations where the system is implemented by an individual e.g. working on a construction site, the individual may move about the environment significantly. Using measurements of sound level characteristics behind and/or outside the acoustic barrier to initialize attenuation tests recognises that in some applications a typical working environment has different sound levels in different places and an individual may move from a region of the environment with lower sound levels, to a region of the environment with higher amplitude noises. Whilst even an incorrectly fitted hearing protection device may provide adequate protection, or no protection may be necessary, in low sound level regions of the environment, adequate protection would be required in high sound level regions of the environment. This would allow for example low power monitoring to be carried out more regularly, or even continuously, but then a more accurate attenuation test using the sound generator to be carried out if the low power monitoring indicates that a poor fit is likely, specifically, in some cases, when a good fit is necessary.

When the processor is not required to compare the test sound produced by the sound generator with the attenuated test sound, the processor may be powered down or operated in a low power mode. Similarly, the microphone and/or the sound generator may only be powered up when the attenuation test is performed. This reduces the power consumption of the system which may increase the lifetime of the system.

The Applicant has appreciated that the principle of carrying out an attenuation test when the conditions (i.e. experienced by the user) indicate that hearing protection is particularly required is novel and inventive in its own right and thus when viewed from a second aspect the invention provides a method for evaluating a fit-effectiveness of a hearing protection device providing an acoustic barrier, comprising:
measuring sound behind the acoustic barrier using a microphone;
determining whether a sound level characteristic of the sound measured by the microphone is above a predetermined threshold;
when the sound level characteristic measured by the microphone is above the predetermined threshold, measuring sound outside the acoustic barrier;
comparing the sound level characteristic of the sound behind and outside the acoustic barrier to determine an attenuation of the hearing protection device;
providing an alert if said determined attenuation is below a cut-off level of attenuation.

The hearing protection device of this aspect of the invention may be provided as part of a system in accordance with the first aspect of the invention and any of the features or optional features thereof may be applicable to this aspect.

This aspect of the invention extends to a hearing protection device arranged to provide an acoustic barrier and comprising a microphone arranged to measure sound behind the acoustic barrier, the hearing protection device being arranged:
to determine whether a sound level characteristic of the sound measured by the microphone is above a predetermined threshold;
when the sound level characteristic measured by the microphone is above the predetermined threshold, to compare the sound level characteristic of the sound behind and outside the acoustic barrier to determine an attenuation of the hearing protection device; and
to provide an alert if said determined attenuation is below a cut-off level of attenuation.

In a set of embodiments the microphone is a first microphone and the hearing protection device comprises a second microphone arranged to measure sound outside the acoustic barrier.

Any of the features or optional features of the first aspect of the invention may be applicable to this aspect of the invention. For example in a set of embodiments the hearing protection device comprises a pair of devices for insertion into respective ear canals provided on a common connecting structure such as a collar.

The Applicant has also appreciated that whilst according to the second aspects of the invention the sound level characteristic measured by the first microphone exceeding the predetermined threshold triggers the comparison of the sound level characteristic of the sound behind and outside the acoustic barrier, in some embodiments the comparison may additionally be triggered by any of the previously discussed stimuli.

For example, in embodiments in which the hearing protection device comprises a position determining arrangement, comparing the measured sound outside the acoustic barrier with the measured sound behind the acoustic barrier may additionally be triggered by a user entering a particular location.

The Applicant has also appreciated that it may not be necessary to implement a system including a sound generator when the system includes a microphone arranged to measure sound behind the acoustic barrier and a second microphone arranged to measure sound outside the acoustic barrier. This is novel and inventive in its own right and thus when viewed from a third aspect the invention provides a system for evaluating an attenuation of a hearing protection device, the system comprising:
a hearing protection device arranged to provide an acoustic barrier;
a first microphone arranged to measure sound behind the acoustic barrier;
a second microphone arranged to measure sound outside the acoustic barrier; and
a processor arranged to compare repeatedly during normal use the measured sound outside the acoustic barrier with the measured sound behind the acoustic barrier to determine an attenuation of the hearing protection device; the system being arranged to provide an alert if the determined attenuation is below a cut-off level of attenuation.

Thus, it will be seen that in accordance with the second and third aspects of the invention, the attenuation of a hearing protection device may be determined by comparing the general sound levels outside of the acoustic barrier in the local environment with the sound levels behind the acoustic barrier. The comparison of the measured sound outside the acoustic barrier with the measured sound behind the acoustic barrier comprises an attenuation test.

As previously described in relation to the first aspect of the invention, the attenuation test may be carried out repeatedly over the lifetime of the device by carrying it out at least once during every period in which the protection device should be used—e.g. every shift for an industrial worker. Advantageously however the attenuation test is carried out repeatedly during a given period of use.

Any of the previously discussed stimuli could trigger the attenuation test but in a set of embodiments the system is arranged to carry out an attenuation test when a measured sound level characteristic behind the acoustic barrier exceeds a threshold. In such embodiments, the attenuation test may be initiated when the sound levels in the local environment become sufficiently loud that damage may be caused to an individual's hearing if the hearing protection device is not fitted correctly. Advantageously, no interaction is required by the individual or by another user (e.g. a manager) for the attenuation test to be carried out. The attenuation test in may therefore be automatic.

In other embodiments the system is arranged to carry out an attenuation test when a measured sound level characteristic outside the acoustic barrier exceeds a threshold.

In accordance with the first and third aspects of the invention and embodiments of the second aspect of the invention, the system or hearing protection device is arranged to provide an alert if the determined attenuation is below the cut-off level of attenuation. Whilst the Applicant has appreciated there may be embodiments in which the cut-off level of attenuation varies e.g. according to the level of the sound experienced in the environment in which the system or hearing protection device is placed, preferably the cut-off level of attenuation is predetermined. This may be the easiest to implement and to manage in practice and will typically enable consistently meeting specified standards.

Whilst the alert referred to herein may be also or instead be provided to another individual or system (such as a manager or central controller), preferably at least one alert is provided to the user (i.e. the individual fitted with the hearing protection device). This is advantageous as it allows the user to be directly aware of the protection they are receiving so that they can take necessary corrective action as soon as required.

The alert to the user may be provided in any suitable form. In a set of embodiments, the alert is a visible indication e.g. provided by a light emitting diode, a flashing light or a colour changing light. In another set of embodiments, the indication is an audio indication e.g. a buzzer or a voice message. This could be delivered to the user behind the acoustic barrier—e.g. with a suitable speaker. In another set of embodiments, the indication is a haptic indication e.g. a vibration. The haptic indication is particularly beneficial in bright, and/or noisy environments, as the vibrations can be felt on the skin of the individual and thus make it easier to perceive the alert.

In order to provide the alert, the system may comprise a further device. This further device may be a smart-watch or tracker which can produce sounds, lights or vibrations. In embodiments in which the system comprises a common connecting structure, the alert may be provided by the common connecting structure. For example, the common connecting structure may produce sounds, lights or vibrate to provide an alert. The further device may be a mobile device such as a smart phone. In such an embodiment, the indication may be in the form of an alert or alarm on the mobile device. The mobile device may carry out other functions of the system described above.

In a set of embodiments, the event referred to above comprises a vocalisation by the user using the hearing protection device. The vocalisation required to trigger the attenuation test may be a unique sound e.g. a specific vowel sound such as ‘eee’ or humming, e.g. performed uninterrupted for a minimum length of time, so that the attenuation test is not triggered unnecessarily by noise in the local environment of the system. Vowel sounds such a ‘eee’ are generally high in energy (between 200-500 Hz) and this makes such sounds more distinguishable from the noise in the local environment. Using a specific vocalisation may help the user easily to control when the attenuation test is performed without requiring use of their hands. For example, a user may notice the noise levels have increased, and use the system to determine whether this is due to the hearing protection no longer being correctly fitted even if their hands are dirty, gloved, or otherwise occupied. However, the Applicant has also appreciated that the user does not need to consciously produce a vocalisation with the intention of triggering an attenuation test. For example, the attenuation test may be triggered by a specific vowel sound such as ‘eee’ which occurs when the user simply speaks during use. Therefore, the attenuation test may be triggered when the user communicates with others in their vicinity. This may allow the attenuation test to be carried out more unobtrusively than when the user is relied upon to consciously make a specific vocalisation to trigger the test. The vocalisation may be detected by the microphone arranged to measure sound behind the acoustic barrier.

The Applicant has recognised that using a specific vocalisation (e.g. in the frequency range 200-500 Hz) as the event can be beneficial since when hearing protection is fitted, such vocalisation can produce louder sounds behind the acoustic barrier (especially where the device comprises an earplug) than outside it through what is known as the occlusion effect.

In fact the Applicant has further appreciated that the occlusion effect can itself be used to perform a test as to whether hearing protection is adequately fitted and thus when viewed from a fourth aspect the invention provides a system for evaluating a fit effectiveness of a hearing protection device inserted into the ear canal of a mammalian subject to provide an acoustic barrier, the system further comprising:
a first microphone arranged to measure sound behind the acoustic barrier;
a second microphone arranged to measure sound outside the acoustic barrier; and
a processor arranged, during normal use, to compare a sound produced during vocalisation of the mammalian subject measured behind the acoustic barrier to the sound measured outside the acoustic barrier, the system being arranged to provide an alert unless a sound level characteristic of the sound measured behind the acoustic barrier is higher than the sound level characteristic of the sound measured outside the acoustic barrier by more than a threshold amount.

In a set of embodiments, the sound level characteristic is measured at a specific frequency or a range of frequencies. For example, the specific frequency may be within the frequency range 200-500 Hz. Within this frequency range vocalisation can produce louder sounds behind the acoustic barrier than outside it. The sound level characteristic may also be measured at a plurality of specific frequencies e.g. within this range.

Certain embodiments of the present invention will now be described, by way of example only, with reference to the accompanying drawings in which:

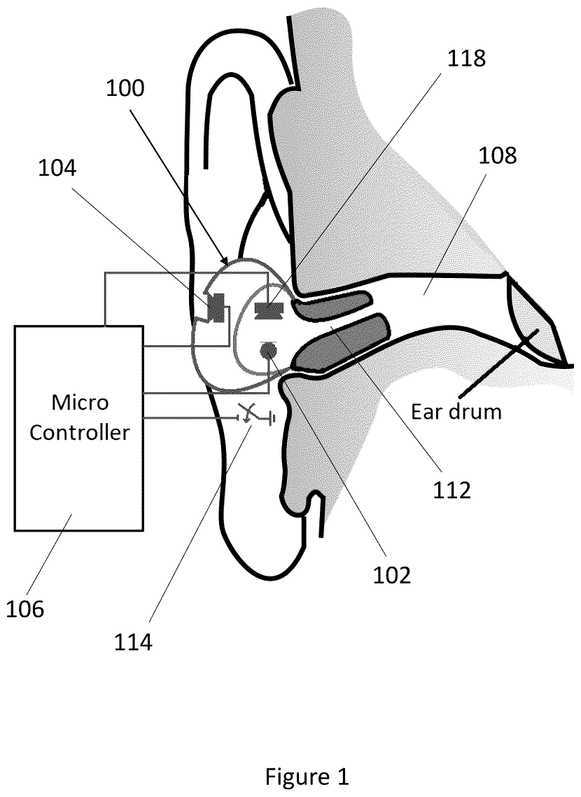

FIG. 1 shows schematically an embodiment of an in-ear hearing protection device in accordance with the invention;

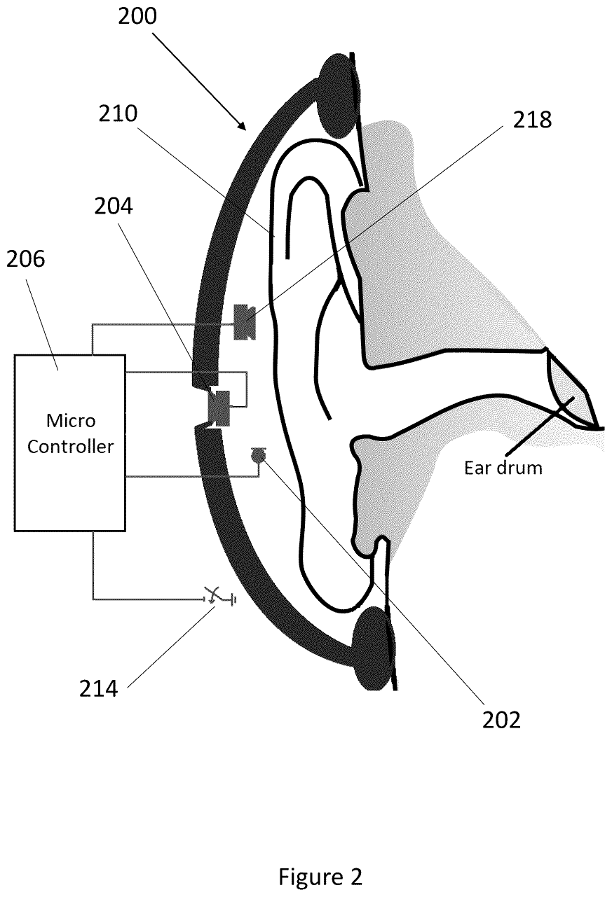

FIG. 2 shows schematically an embodiment of an over-the-ear hearing protection device in accordance with the invention;

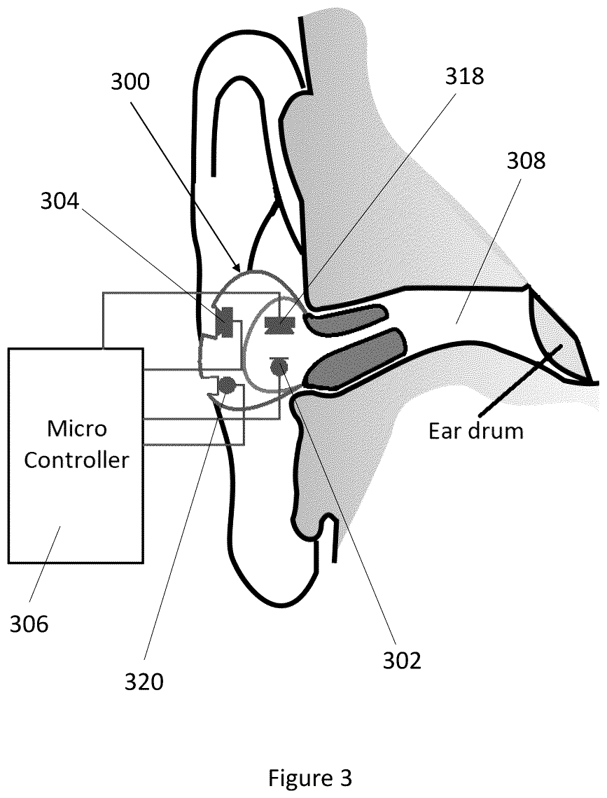

FIG. 3 shows schematically an embodiment of another in-ear hearing protection device in accordance with the invention;

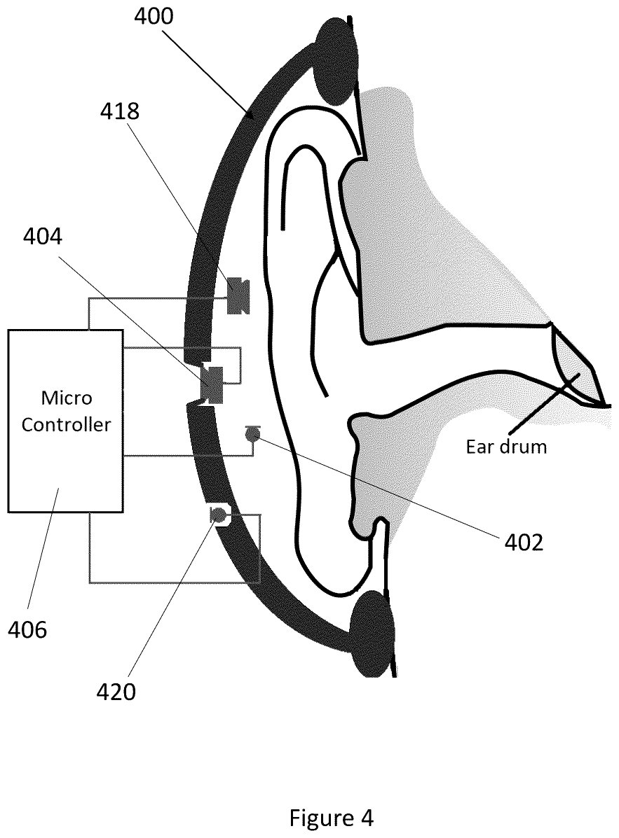

FIG. 4 shows schematically an embodiment of another over-the-ear hearing protection device in accordance with the invention;

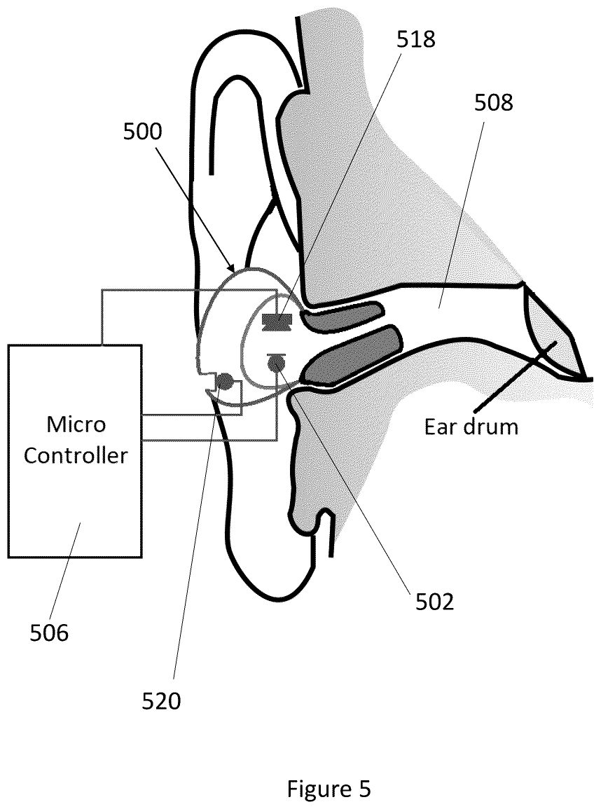

FIG. 5 shows schematically an embodiment of another in-ear hearing protection device in accordance with the invention;

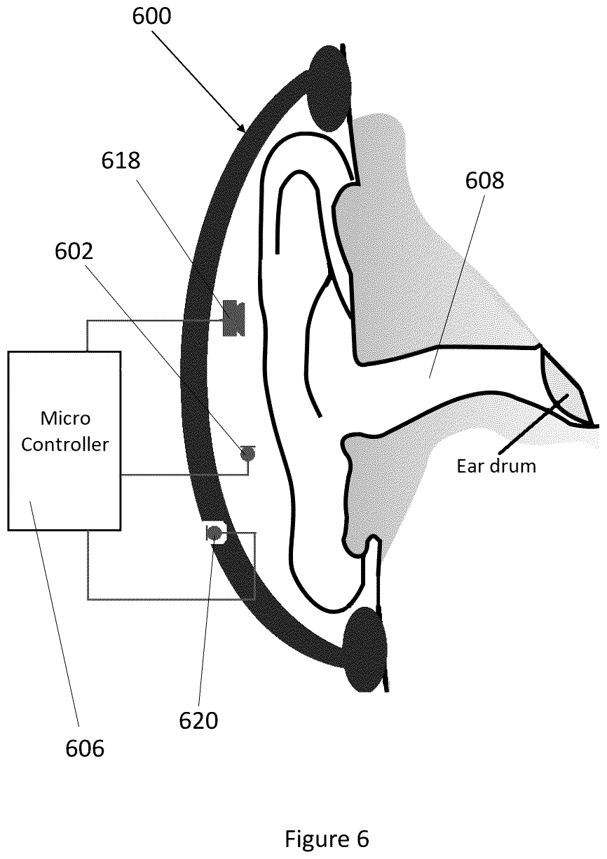

FIG. 6 shows schematically an embodiment of another over-the-ear hearing protection device in accordance with the invention;

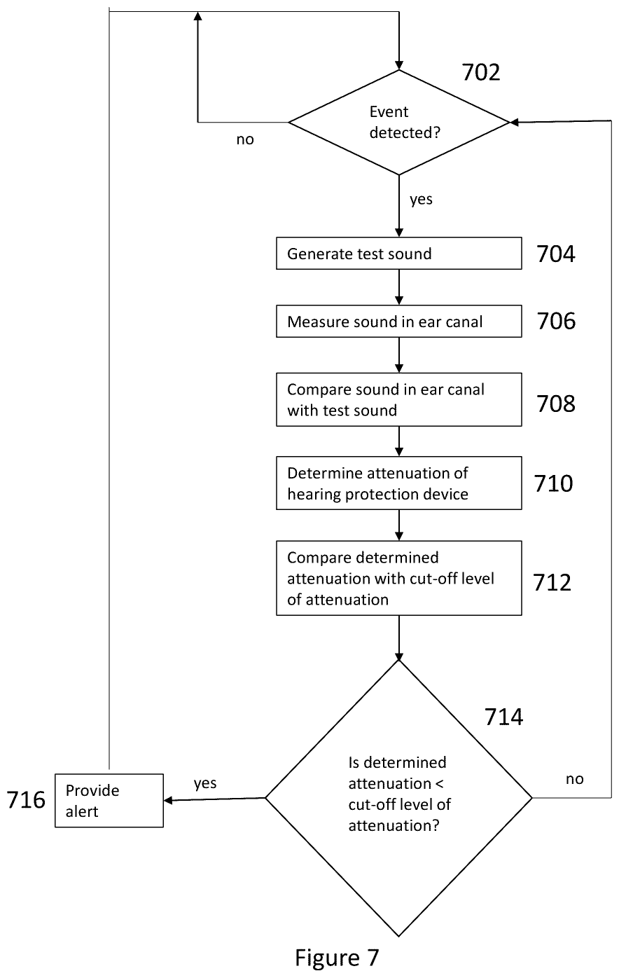

FIG. 7 is a flow chart demonstrating an exemplary attenuation test;

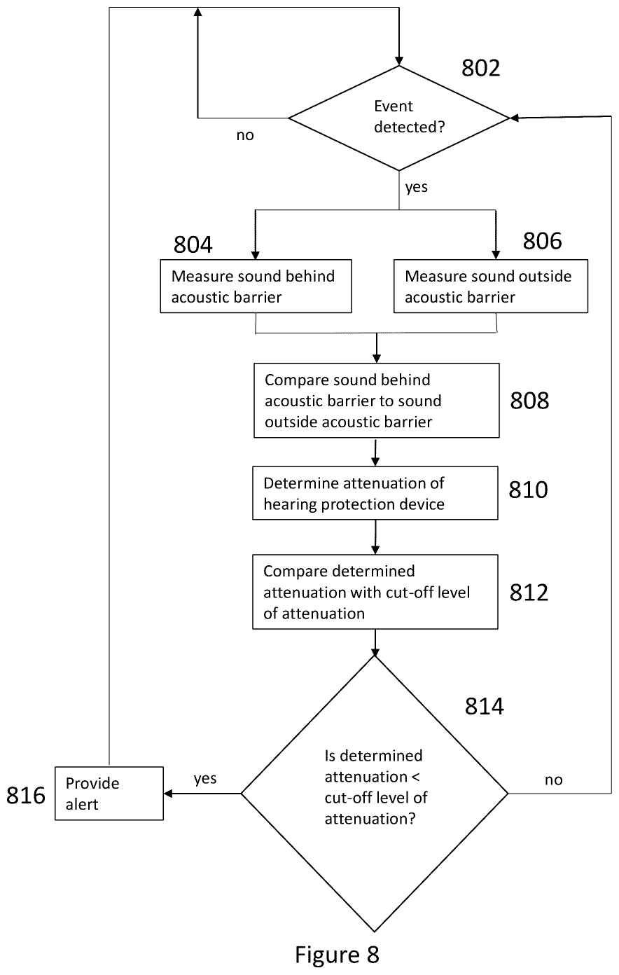

FIG. 8 is a flow chart demonstrating another exemplary attenuation test which does not require a sound generator;

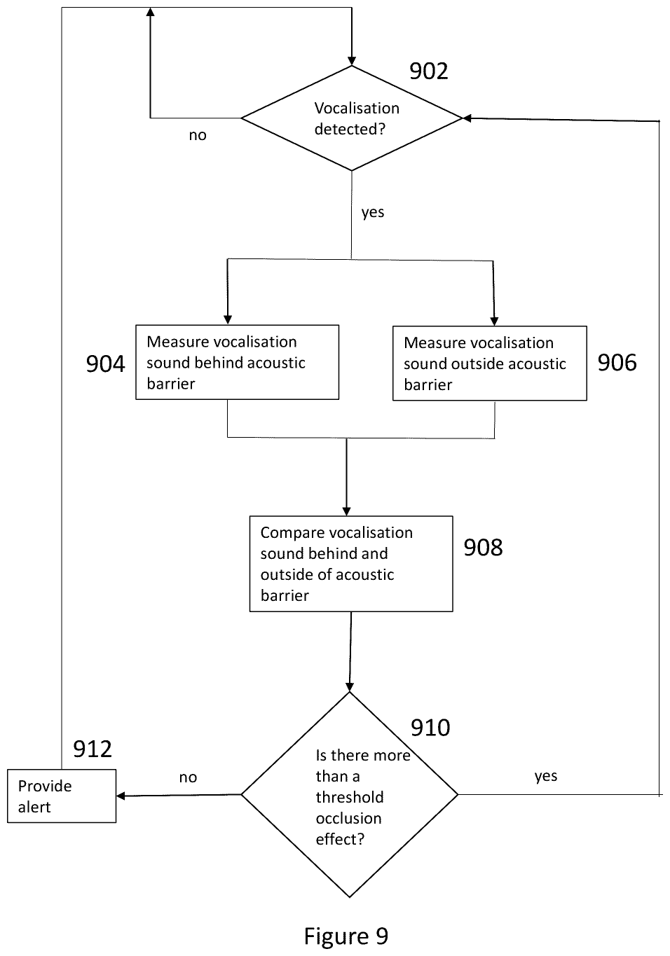

FIG. 9 is a flow chart demonstrating another exemplary attenuation test using the occlusion effect; and

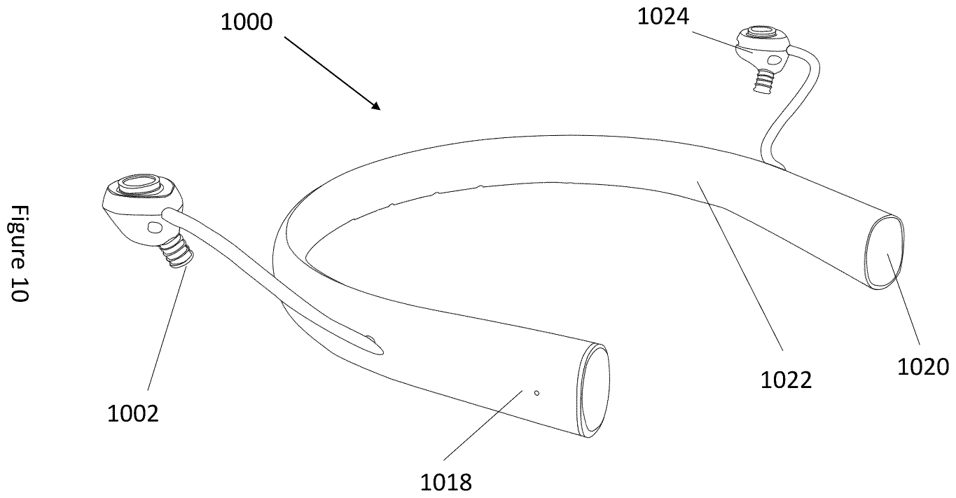

FIG. 10 shows schematically another embodiment of a hearing protection device in accordance with the invention.

FIG. 1 shows a schematic cross-section of an in-ear hearing protection device or earplug 100 in accordance with the invention which is inserted into the ear canal 108 of an individual. Therefore, the earplug 100 provides an acoustic barrier which attenuates sounds from the local environment (e.g. outside of the ear) so that the ear drum of the individual is subjected to reduced amplitude sounds. Whilst a singular earplug 100 is shown in FIG. 1, the individual is likely to use earplugs in both of their ears to provide protection against damage to their hearing.

The position of the earplug 100 shown in FIG. 1 is the correctly fitted position. In this configuration, the earplug 100 is fully inserted so that it creates a seal within the ear canal 108. There are no air pockets between the earplug 100 and the walls of the ear canal 108. In this position the level of attenuation provided by the earplug 100 is approximately the intended level of attenuation that can be provided by the earplug 100. In this correctly fitted position, the optimum protection against damage is provided.

The earplug 100 comprises a sound generator 104. The sound generator 104 is arranged to generate a test sound of known characteristics such as frequency, amplitude and duration. Although the sound generator is provided somewhat within the earplug 100, an acoustic channel is provided (not shown) so that the test sound is directed into the local environment outside of the earplug 100. The earplug 100 attenuates this test sound produced by the sound generator 104 alongside other sounds from the local environment.

The earplug 100 further comprises a microphone 102. The microphone 102 is arrange to detect sound in the ear canal 108, behind the acoustic barrier provided by the earplug 100. As shown in FIG. 1, the microphone is connected to the inside of the ear canal 108 by a channel 112 in the earplug 100. Sound which penetrates through the acoustic barrier formed by the earplug 100 passes into the ear canal 108 and propagates along the acoustic path provided by the channel 112. It is also is detected by the microphone 102.

The microphone 102 and sound generator 104 are connected to a controller 106 which includes a processor. This could be provided as an integral part of the earplug 100 or the controller 106 could be separately provided e.g. by a mobile device (e.g. a mobile phone) carried by the individual using the earplug 100. The controller 106 is also connected to a loudspeaker 118 arranged to produce sound behind the acoustic barrier provided by the earplug.

There are a number of ways in which the attenuation test may be initiated. As seen in FIG. 1, the earplug 100 of this embodiment comprises a button 114. The button 114 is configured to initiate the sound generator 104 producing the test sound, the detection of the sound in the ear canal by the microphone 106, the determination of the attenuation provided by the earplug 100 and the comparison of the cut-off level of attenuation to the determined attenuation. In other words the attenuation test is initiated by pressing the button 114. This provides a straightforward mechanism for enabling the test to be carried out at an appropriate time—e.g. at the beginning of a shift, when prompted upon entering a potentially hazardous area, when requested by a manager, or simply if the user perceives that the sound they are experiencing is too loud. However, there are many possible alternative ways to initiate the test—such as when the earplug 100 receives an external signal, when the user is determined to have entered a potentially hazardous zone by a position system or when the level of sound detected by the internal microphone 102 exceeds a threshold.

Operation of the device will now be described with additional reference to FIG. 7. When it is determined that a suitable event indicates that an attenuation test should be performed (step 702), the controller 106 causes the sound generator 104 to generate a test sound of known characteristics (step 704). This is measured by the microphone 102 (step 706) and the processor in the controller 106 compares one or more of the known sound characteristics of the test sound produced by the sound generator 104 to the sound measured by the microphone 102 (step 708). This comparison may involve subtracting the amplitude of the sound measured by the microphone 102 from the known amplitude of the test sound generated by the sound generator 104. The controller 106 uses the result of the comparison to determine the attenuation of the earplug 100 (step 710). The result of the comparison may directly yield the attenuation of the earplug 100, or the controller 106 may use algorithms and/or comparison tables to determine a numerical value corresponding to the attenuation.

The controller 106 then compares the determined attenuation of the earplug 100 to a cut-off level of attenuation of the earplug 100 which is stored in its memory (step 712). This comparison may involve subtracting the determined attenuation from the cut-off level of attenuation. At step 714, if the determined attenuation of the earplug 100 is determined to be below the cut-off level of attenuation of the earplug 100 (e.g. the subtraction of the determined attenuation from the cut-off level of attenuation yields a positive number), then an alert is provided to the individual (step 716). The cut-off level of attenuation may be defined as the minimum level of attenuation the earplug 100 must provide to provide adequate protection against permanent damage to an individual's hearing.

In this embodiment the alert is provided by a tone generated by the loudspeaker 118, which is located behind of the acoustic barrier provided by the earplug 100. The alert indicates to the user that the earplug 100 is providing an attenuation of sound below the cut-off level of attenuation of the earplug 100. Therefore, the alert may indicate to the user that the earplug 100 is not correctly fitted, and therefore the earplug 100 should be adjusted or refitted. The alert may cease when the difference between the determined attenuation of the earplug 100 is no longer below the cut-off level of attenuation of the earplug 100.

Other forms of alert may be provided in addition or instead. In particular it may be necessary to provide another form of alert to account for the situation where the user has not fitted the hearing protection device 100 at all. For example, the alert may be provided by an alternative means such as a notification on a mobile device belonging to the individual or a haptic vibration provided by an external device worn by the user.

When an individual is using the earplug 100 over an extended period of time, it is desirable that the attenuation provided by the earplug 100 is repeatedly determined and compared to the cut-off level of attenuation of the earplug 100. This is because the earplug 100 may have become dislodged from its correctly fitted position over time, e.g. as an individual performs physical task and moves around their environment. This individual may also remove the earplug 100 to communicate with others and then replace the earplug 100 in an incorrect position or forget to do so at all.

FIG. 2 shows schematically an exemplary cross-section of one of a pair of over-the-ear defenders 200. The over-the-ear defender 200 enclose the entire outer ear 210 of an individual. The outer layer 201 of the over-the-ear defender 200 is soft so that forms a seal against the user's head to provide an acoustic barrier to reduce the sound pressure at their ear drum. The over-the-ear defender 200 comprises a microphone 202, a sound generator 204 and a controller 206 which performs a similar role to the controller in the first embodiment. As before, the controller 206 may be provided integrally with the over-the-ear defender 200 as indicated in FIG. 2. However, in other embodiments it may be located elsewhere e.g. in a mobile device carried by the individual. The over-the-ear defender 200 may also comprise a button 214 and a loudspeaker 218. These components perform similar functions to those previously described in relation to the earplugs 100 seen in FIG. 1.

FIG. 3 shows an exemplary cross-section of an earplug 300 for insertion into the ear canal 308 of an individual in accordance with another embodiment. The earplug 300 comprises a plurality of identical components to those seen in earplug 100 (in FIG. 1), namely: an (internal) microphone 302, a sound generator 304, a controller 306 and a loudspeaker 318. These components perform the functions previously described in relation to FIG. 1.

The earplug 300 as seen in FIG. 3 however additionally comprises an external microphone 320. Whilst in FIG. 3 the external microphone 320 is located in the structure of the earplug 300 (e.g. the external microphone 320 could be located on a common PCB with the (internal) microphone 302), in other embodiments not shown where the external microphone 320 is provided separately, e.g. in a mobile device.

The external microphone 320 is arranged to measure the sound outside of the earplug 300, i.e. in the local environment outside of the earplug 300. The external microphone 320 can therefore be considered to measure the ambient sound that the ear of an individual would be subjected if the earplug 300 was not fitted. In FIG. 3, the external microphone 320 shown as being located externally of the acoustic barrier provided by the earplug 300. However, the external microphone 320 could by physically located behind the acoustic barrier provided by the earplug 300. A channel may then be provided such that sounds from outside of the acoustic barrier can be detected by the external microphone 320.

The external microphone 320 can be used to measure the test sound produced by the sound generator 304. The external microphone 320 can therefore be used to determine the sound characteristic of the test sound. This may then be used to confirm the accuracy of the known sound characteristic of the test sound which is compared to the sound characteristic of the sound detected by the internal microphone 302 i.e. the attenuated test sound. Alternatively, the sound characteristic determined using the external microphone 320 may be compared to the attenuated test sound. This comparison is then used to determine the attenuation provided by the earplug 300. The external microphone 320 can also be used to measure the environmental sound pressure outside the earplug 300. This might be measured regularly or even continuously.

When the sound pressure measured by the internal microphone 302 exceeds a threshold level, this initiates the attenuation test. Thus as previously described, the sound generator 304 produces a test sound, the sound is measured in the ear canal by the microphone 302, and a determination of the attenuation of the earplug 300 is carried out by comparing the cut-off level of attenuation to the determined attenuation. Should the determined actual attenuation be below the cut-off level of attenuation, an alert is then provided to the user. Thus, the microphone 302 enables an attenuation test to be repeated during use of the earplugs 300 when the sound levels in the ear canal exceed a threshold. This threshold may correspond to sound levels in the local environment being capable of causing damage to hearing if the earplugs 300 are not correctly fitted. The advantage of using the local sound level to initiate the test is that it enables testing to take place exactly when protection is needed.

FIG. 4 shows another embodiment of an over-the-ear defender 400 with an external microphone 420. The other features are similar to those of the embodiment of FIG. 2 with a ‘4’ prefix instead of ‘2’.

FIG. 5 shows an exemplary cross-section of an earplug 500 for insertion into the ear canal 508 of an individual in accordance with another embodiment. The earplug 500 comprises a plurality of identical components to those seen in earplug 300 (in FIG. 1), namely: an (internal) microphone 502, a controller 506, a (internal) loudspeaker 518 and an external microphone 520. These components perform the functions previously described in relation to FIG. 3.

The earplug 500 shown in FIG. 5 however does not include an external sound generator as seen in FIG. 3. The external microphone 520 is used to measure an environmental sound level characteristic such as sound pressure outside the earplug 500 and the (internal) microphone 502 is used to measure the sound pressure behind the earplug 500. When the sound pressure measured by the (internal) microphone 502 exceeds a threshold level, this initiates an attenuation test which involves comparing the measured sound pressure outside the earplug 500 to the measured sound pressure behind the earplug 500.

Operation of the device shown in FIG. 5 will now be described with additional reference to FIG. 8. When a suitable event is detected (step 802), this indicates that an attenuation test should be performed. A suitable event could be the (internal) microphone 502 measuring a particular sound level characteristic, e.g. sound pressure, which exceeds a threshold. The internal microphone 502 measures one or more sound level characteristics, e.g. the sound pressure, in the ear canal 508 (step 804). The external microphone 520 measures the corresponding one or more sound level characteristics outside of the earplug 500 (step 806). Step 804 and step 806 are typically carried out simultaneously, to ensure that the sounds detected by both the internal microphone 302 and the external microphone 320 are caused by the same environmental noise. The controller 506 then compares the measured sound level characteristic(s) outside of the earplug 500 with the measured sound level characteristic(s) inside of the earplug 500 (step 808). The controller 506 uses the result of the comparison to determine the attenuation of the earplug 500 (step 810). The result of the comparison may directly yield the attenuation of the earplug 500, or the controller 506 may use algorithms and/or comparison tables to determine a numerical value corresponding to the attenuation.

The controller 506 then compares the determined attenuation of the earplug 500 to a cut-off level of attenuation of the earplug 500 which is stored in its memory (step 812). This comparison may involve subtracting the determined attenuation from the cut-off level of attenuation. At step 814, if the determined attenuation of the earplug 500 is determined to be below the cut-off level of attenuation of the earplug 500 (e.g. the subtraction of the determined attenuation from the cut-off level of attenuation yields a positive number), then an alert is provided to the individual (step 816). The cut-off level of attenuation may be defined as the minimum level of attenuation the earplug 500 must provide to provide adequate protection against permanent damage to an individual's hearing.

In this embodiment the alert is provided by a tone generated by the loudspeaker 518, which is located behind the acoustic barrier provided by the earplug 500. The alert indicates to the user that the earplug 500 is providing an attenuation of sound below the cut-off level of attenuation of the earplug 500. Therefore, the alert may indicate to the user that the earplug 500 is not correctly fitted, and therefore the earplug 500 should be adjusted or refitted. The alert may cease when the difference between the determined attenuation of the earplug 500 is no longer below the cut-off level of attenuation of the earplug 500. Other forms of alert may be provided in addition or instead. Whilst a loudspeaker 518 is shown in FIG. 5, should another form of alert be provided then the loudspeaker may not be required.

It will also be appreciated that the method shown in FIG. 7 could be implemented in any of the devices shown in FIGS. 3, 4 and 6.

FIG. 6 shows another embodiment of an over-the-ear defender 600 with an (internal) microphone 602 and external microphone 620, but without a sound generator. The other features are similar to those of the embodiment of FIG. 2 with a ‘6’ prefix instead of ‘2’.

FIG. 10 shows another embodiment of a hearing protection device 1000. The hearing protection device 1000 includes a pair of earplugs 1024. The pair of ear plugs 1024 are connected by a common connecting structure, i.e. a collar 1002. The earplugs 1024 provide an acoustic barrier to attenuate sounds from the local environment. The earplugs 1024 include an (internal) microphone, arranged to detect sound in the ear canal, behind the acoustic barrier provided by the earplug 1024.

The collar 1002 is arranged to rest around the neck and shoulders of a user. The collar 1002 includes a loudspeaker 1018 and an external microphone 1020. The external microphone 1020 is arranged to measure the sound outside of the earplugs 1024.

The loudspeaker 1018 is arranged to provide an alert, e.g. in the form of a tone, to the user and/or other individuals in the local area, indicating that the earplugs 1024 is providing a level of attenuation below the cut off level of attenuation. However, it is also possible for the loudspeaker 1018 to be used as a sound generator, e.g. to create a test sound.

Whilst the external microphone 1020 and the loudspeaker 1018 are mounted on the collar in the embodiment shown in FIG. 10, it will be appreciated that in other embodiments the external microphone 1020 and/or the loudspeaker 1018 may be mounted on the ear plug 1024 (e.g. similarly to the arrangement shown in FIGS. 1 and 3).

The device 1000 shown in FIG. 10 may be operated in accordance with the steps shown in FIG. 8. It will be appreciated that in embodiments in which the loudspeaker 1018 used as a sound generator, that the hearing protection device may also operate in accordance with the steps shown in FIG. 7.

In a set of embodiments set out hereinabove, the attenuation test is carried out by measuring sound behind the acoustic barrier derived from the test sound produced by the sound generator or the ambient sound from the environment. However in other embodiments an attenuation test is carried out using the so-called occlusion effect whereby vocalisations made by an individual are louder in the ear canal when the hearing protection device earplug is being worn. This will now be described with reference to FIG. 9.

This method may be performed using either the earplug 300 shown in FIG. 3, the over-the-ear defender 400 shown in FIG. 4 or the hearing protection device 1000 shown in FIG. 10. It should however be noted that sound generators 304, 404 are not required to perform the method set out in FIG. 9. For clarity, the method shown in FIG. 9 will be described in relation the system shown in FIG. 3.

As mentioned above the method described hereinbelow uses a user's vocalisation to carry out the attenuation test. The test can be prompted by any of the stimuli discussed previously, but equally detection of the vocalisation (e.g. a specific extended vowel sound) can be used as the trigger. When a vocalisation (i.e. the event) is detected (e.g. by either the internal microphone 302 or the external microphone 320), this indicates that an attenuation test should be performed (step 902). The internal microphone 302 measures one or more sound level characteristics of the vocalisation sound, e.g. the sound pressure produced by the vocalisation, in the ear canal 308 at certain frequencies (step 904). The external microphone 320 measures the corresponding one or more sound level characteristics of the vocalisation sound outside of the earplug 300 (step 906) at certain frequencies. Step 904 and step 906 are typically carried out simultaneously, to ensure that the same vocalisation pattern is detected by both the internal microphone 302 and the external microphone 320.

The processor in the controller 306 then compares the sound level characteristics of the vocalisation measured in the ear canal to the sound level characteristics of the vocalisation measured outside the ear canal (step 908).

In step 910, the processor in the controller 306 determines whether a significant occlusion effect is present. An occlusion effect is present when a certain aspect of the vocalisation sound pressure (e.g. the vocalisation sound pressure at certain frequencies) measured in the ear canal 308 is higher than the certain aspect of the vocalisation sound pressure measured outside the earplug 300. The presence of a sufficient degree of occlusion indicates that the earplug 300 is correctly fitted. The absence of sufficient occlusion, e.g. where the certain aspect of the vocalisation sound pressure measured in the ear canal 308 is lower than the certain aspect of the vocalisation sound pressure measured outside the earplug 300, or the certain aspect of the vocalisation sound pressure measured in the ear canal 308 is higher than the certain aspect of the vocalisation sound pressure measured outside the earplug 300 but not sufficiently higher, indicates that the earplug 300 is not correctly fitted.

In step 912 an alert is provided to the individual if the certain aspect of the vocalisation sound pressure measured in the ear canal 308 is not higher than the certain aspect of the vocalisation sound pressure outside the earplug 300 by more than a threshold amount. The threshold is preferably defined such that the alert is given if the earplug 300 is not fitted well enough to avoid a risk of damage to the individual's hearing.

In this embodiment the alert is provided by a tone generated by the loudspeaker 318 as previously described. It may be provided in any of the ways previously described however.

The method steps 902-912 may be repeated any number of times during use of the hearing protection by the individual.

Thus it will be seen that a number of specific examples have been described which illustrate how the invention may be implemented to allow regular testing of how effectively hearing protection has been fitted and indeed whether it has been fitted at all. This has significant potential to increase the safety of workers and others in high noise environments by limiting the risk of long-term hearing damage. There are many different variants and combinations of the features discussed herein which could be employed depending on the requirements of the particular application. The invention is thus not to be considered to be limited by any of the examples shown.

1. A hearing protection device arranged to provide an acoustic barrier and comprising a microphone arranged to measure sound behind the acoustic barrier, the hearing protection device being arranged:
to determine whether a sound level characteristic of the sound measured by the microphone is above a predetermined threshold;
when the sound level characteristic measured by the microphone is above the predetermined threshold, to compare the sound level characteristic of the sound behind and outside the acoustic barrier to determine an attenuation of the hearing protection device; and
to provide an alert if said determined attenuation is below a cut-off level of attenuation.

2. A hearing protection device as claimed in claim 1 comprising a second microphone arranged to measure sound outside the acoustic barrier.

3. A system for evaluating an attenuation of a hearing protection device, the system comprising:
a hearing protection device arranged to provide an acoustic barrier;
a first microphone arranged to measure sound behind the acoustic barrier;
a second microphone arranged to measure sound outside the acoustic barrier; and
a processor arranged to compare repeatedly during normal use the measured sound outside the acoustic barrier with the measured sound behind the acoustic barrier to determine an attenuation of the hearing protection device;
the system being arranged to provide an alert if the determined attenuation is below a cut-off level of attenuation.

4. The system as claimed in claim 3, arranged to compare the measured sound outside the acoustic barrier with the measured sound behind the acoustic barrier when a measured sound level characteristic behind the acoustic barrier or outside the acoustic barrier exceeds a threshold.

5. The system as claimed in claim 3 or 4, further comprising a mobile device.

6. The system, as claimed in claim 5, wherein the mobile device is arranged to provide the alert.

7. The hearing protection device, or system, as claimed in any of claims 2 to 6, wherein the hearing protection device comprises a pair of devices for insertion into respective ear canals mounted to a common connecting structure and wherein the second microphone is provided on the common connecting structure.

8. The hearing protection device, or system, as claimed in claim 7, wherein the common connecting structure is arranged to provide the alert.

9. The hearing protection device, or system, as claimed in any preceding claim, wherein the hearing protection device comprises a device for insertion into an ear canal.

10. The hearing protection device, or system, as claimed in any preceding claim, comprising a position determining arrangement and additionally arranged to determine or evaluate said attenuation upon a user entering a particular location.

11. The hearing protection device, or system, as claimed in any proceeding claim, arranged to provide the alert to an individual fitted with the hearing protection device.

12. A method for evaluating a fit-effectiveness of a hearing protection device providing an acoustic barrier, comprising:
measuring sound behind the acoustic barrier using a microphone;
determining whether a sound level characteristic of the sound measured by the microphone is above a predetermined threshold;
when the sound level characteristic measured by the microphone is above the predetermined threshold, measuring sound outside the acoustic barrier;
comparing the sound level characteristic of the sound behind and outside the acoustic barrier to determine an attenuation of the hearing protection device;
providing an alert if said determined attenuation is below a cut-off level of attenuation.

13. A system for evaluating an attenuation of a hearing protection device, the system comprising:
a hearing protection device arranged to provide an acoustic barrier;
a microphone arranged to measure sound behind the acoustic barrier;
a sound generator arranged to generate a test sound outside the acoustic barrier with a known sound characteristic; and
a processor arranged to compare repeatedly during normal use the test sound generated by the sound generator with the measured sound behind the acoustic barrier to determine an attenuation of the hearing protection device;
the system being arranged to provide an alert if the determined attenuation is below a cut-off level of attenuation.

14. The system as claimed in claim 13, wherein the sound generator is provided on the hearing protection device.

15. The system as claimed in claim 13 or 14, further comprising a mobile device, wherein the mobile device provides the processor.

16. The system as claimed in any of claims 13 to 15, wherein the microphone is arranged to measure a sound level characteristic behind the acoustic barrier and the system is arranged to determine said attenuation when said sound level characteristic behind the acoustic barrier exceeds a threshold internal sound level characteristic.

17. The system as claimed in any of claims 13 to 16, further comprising a second microphone arranged to measure sound outside the acoustic barrier.

18. The system as claimed in claim 17, wherein the second microphone is arranged to measure the test sound generated by the sound generator.

19. The system as claimed in claim 18, wherein the processor is arranged to compare the measured sound behind the acoustic barrier to the measured sound outside of the acoustic barrier when the sound generator is operative.

20. A system for evaluating a fit effectiveness of a hearing protection device inserted into the ear canal of a mammalian subject to provide an acoustic barrier, the system further comprising:
a first microphone arranged to measure sound behind the acoustic barrier;
a second microphone arranged to measure sound outside the acoustic barrier; and
a processor arranged, during normal use, to compare a sound produced during vocalisation of the mammalian subject measured behind the acoustic barrier to the sound measured outside the acoustic barrier, the system being arranged to provide an alert unless a sound level characteristic of the sound measured behind the acoustic barrier is higher than the sound level characteristic of the sound measured outside the acoustic barrier by more than a threshold amount.

21. A system as claimed in claim 20, wherein the sound level characteristic is measured within the frequency range 200-500 Hz.
